(19) United States
(12) Patent Application Publication
(10) Pub. No.: US 2025/0375577 A1
(43) Pub. Date: Dec. 11, 2025

(54) NEEDLE UNCAPPING DEVICE

(71) Applicant: Merck Sharp & Dohme LLC, Rahway, NJ (US)

(72) Inventors: Derrick Matthew Smith, Newtown Square, PA (US); Paoly Lao, King of Prussia, PA (US)

(21) Appl. No.: 18/737,001

(22) Filed: Jun. 7, 2024

(51) Int. Cl. A61M 5/32

(52) U.S. Cl. CPC A61M 5/3278

(57) ABSTRACT

A needle uncapping device enables safe removal and replacement of a syringe cap by securely engaging with the cap positioned in an opening on a top surface of the device, allowing an operator to uncap or recap the syringe with reduced effort and risk of accidental needle stick. When a syringe cap is placed in an appropriately sized opening, and the operator pulls the syringe upward, a lip positioned above the opening holds the cap in place, allowing the operator to expose the needle. The top surface of the device is angled downward and protrudes outward from the device body to shield the operator’s hand, and a base of the body protrudes outward to provide additional stability.

### TECHNICAL FIELD

The subject matter described generally relates to medical instruments, and more specifically, to a needle uncapping device that securely engages with a cap of a syringe and allows the syringe to be safely uncapped and recapped at decreased risk to an operator.

### BACKGROUND

Operators attempting to remove or replace a cap of a syringe risk accidental needle stick and bodily harm if the cap is not securely positioned when the needle is exposed. Existing solutions do not adequately protect the operator’s hand when recapping needles and also pose a risk when the operator attempts to forcibly remove a needle cap.

### BRIEF DESCRIPTION OF THE DRAWINGS

The disclosed embodiments have advantages and features which will be more readily apparent from the following detailed description and the appended claims, when taken in conjunction with the accompanying drawings, in which:

FIG. 1 illustrates a needle uncapping device, according to an example embodiment.

FIG. 2 illustrates a side view of the needle uncapping device of FIG. 1, according to an example embodiment.

FIG. 3 illustrates a top view of the needle uncapping device of FIG. 1, according to an example embodiment.

FIG. 4 illustrates a bottom view of the needle uncapping device of FIG. 1, according to an example embodiment.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described.

A needle uncapping device enables safer removal and replacement of a syringe cap by securely engaging with the cap positioned in an opening on a top surface of the device, allowing an operator to uncap or recap the syringe with reduced effort and risk of accidental needle stick. In various embodiments, the device includes multiple openings of different sizes for use with syringes and caps of various types and sizes.

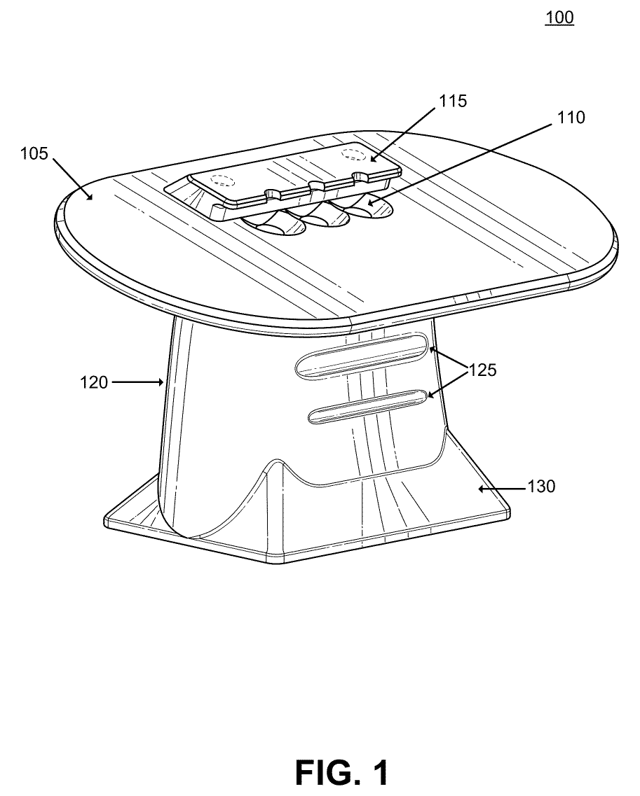

Referring now to FIG. 1, it illustrates a perspective view of an example embodiment of a needle uncapping device 100. In the embodiment shown, the device 100 includes a top planar surface 105 having one or more openings 110 and a ledge 115 with cutouts positioned above and adjacent to each of the openings 110. A lip extends from the base of the ledge 115 outwards toward the openings 110 to allow a cap of a syringe to securely engage with an opening 110, enabling a user of the device 100 to remove the cap and expose the needle.

The top surface 105 is positioned above an elongated body 120 configured to be held by a user of the device 100. For example, as shown in FIG. 1, in some embodiments the body 120 includes grips 125 on the front and rear of the body 120 that are sized to engage with a user’s fingers, allowing the user to hold the device 100 firmly in place when the device 100 is in use (e.g., when the user is removing or reattaching a cap). While the displayed embodiment includes grips 125 on both the front and rear of the body 120, in other configurations, a single grip 125 is positioned on the front or rear of the body or one or more grips 125 are positioned on one or both sides of the body 120.

Moreover, the front and back of the base 130 protrude outward from the body 120 to provide additional stability by increasing the device footprint. In one embodiment, the base 130 protrudes outward at an approximately 40- to 70-degree angle. Still further, in some embodiments, one or more attachment mechanisms are positioned on a bottom planar surface of the base 130, such as an adhesive, suction cups, Velcro, or the like to securely attach the device 100 to an adjacent surface, such as a table or desk.

The top surface 105 of the device 100 is angled downward at an approximately 10-to 30-degree angle toward a user of the device 100 and protrudes outward from the elongated body 120 such that the top surface 105 shields the hand of a user of the device 100. That is, when a user holds the device 100 by placing one hand around the grips 125 of the elongated body 120, the top surface 105 is positioned between the user’s first hand and a syringe held in the user’s other hand. In this way, the top surface 105 acts as a shield to prevent an exposed needle from coming into contact and injuring the hand holding the device 100. In another embodiment, the top surface 105 parallel to the base 130 of the device 100 (i.e., the top surface 105 is not angled downward).

The openings 110 are positioned at an approximate horizontal midpoint of the top surface 105 and above the elongated body 120. In the displayed embodiment, each opening 110 is circular and has a first diameter at the top surface 105 of the device 100 and a second, smaller diameter at a depth below the top surface 105 at a bottom of the opening 110. While the device 100 depicted in FIG. 1 has openings 110 of equal diameters and depths, in other configurations, the device 100 includes openings 110 of different sizes to accommodate different sized syringes and caps. Moreover, while the displayed openings 110 are immediately adjacent to each other, in other embodiments, the openings 110 are spaced apart from each other along the top surface 105. Still further, in some embodiments, the elongated body 120 positioned below the openings 110 is fully or partially hollow, allowing caps to be discarded after use.

While the embodiment displayed in FIG. 1 includes three openings 110, in other embodiments, the device 110 includes fewer or more openings 110 (e.g., one to five). In configurations where the device 110 includes multiple openings 110, each opening 110 may be configured to engage with a needle of a different size. For example, a first opening 110 may have a depth of 30 millimeters (mm), a second opening 110 may have a depth of 50mm, and a third opening may have a depth of 80mm. In various embodiments, the openings 110 have a depth of approximately 30-80mm and a diameter of approximately 6-12mm. The openings 110 may be tapered (e.g., 0.5 degree draft) to help the lever action on the needle cap as the user is positioning the cap under the lip of the ledge 115. Moreover, in some embodiments, the openings 110 are color-coded or otherwise visually distinguished to indicate to the user the size or type of needle or cap that may be used with each respective opening 110.

The needle uncapping device 100 may be made of nylon, acrylonitrile butadiene styrene (ABS) plastic, thermoplastic polymer (such as polyethylene, polypropylene, or polytetrafluoroethylene), or another rigid material and may be manufactured using injection molding, panel forming, blow molding, thermoforming, 3D printing, or the like. The needle uncapping device 100 may be constructed in a single piece or in multiple pieces. For example, in one example embodiment, the top surface 105 is constructed separately from the body 120. In another example embodiment, the needle uncapping device 100 is constructed in multiple pieces and assembled (e.g., using vacuum molding to bond together the individual pieces).

In one example embodiment, the top surface 105 of the device is approximately four inches by eight inches and is approximately two inches wider than the body 120. To accommodate users having varying hand sizes, the height of the body 120 is approximately three to four inches in the front and four to five inches in the back. In other embodiments, the dimensions of the various portions of the device 100 are different to enable engagement with different types, sizes, and number of needles. In general, the device 100 may be configured to engage with any type of syringe.

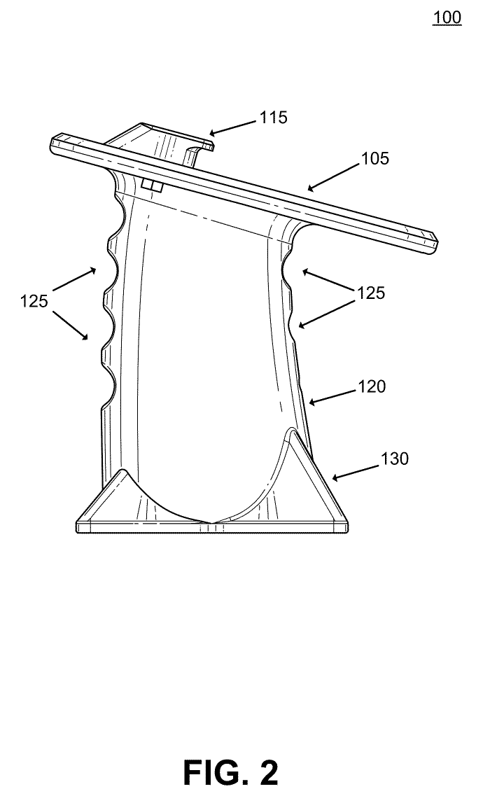

FIG. 2 illustrates a side view of the needle uncapping device 100 of FIG. 1, according to an example embodiment. As shown in the displayed embodiment, the device 100 includes grips 125 on both the front and rear of the body 120 and a base 130 protruding outward from a bottom portion of the body 120 to provide stability when the device 100 is in use and decrease the likelihood of an accidental needle stick by a user. Moreover, the displayed embodiment illustrates the lip extending outward from a front of the ledge 115 and positioned above and adjacent to the openings 110 on the top surface 105 of the device 100 such that the lip securely engages with the cap of a syringe placed in an opening 110. When the user places the top of the cap in an opening 110 and pulls the syringe upward, the lip holds the cap in place in the opening 110 allowing the user to expose the needle and recap it when desired.

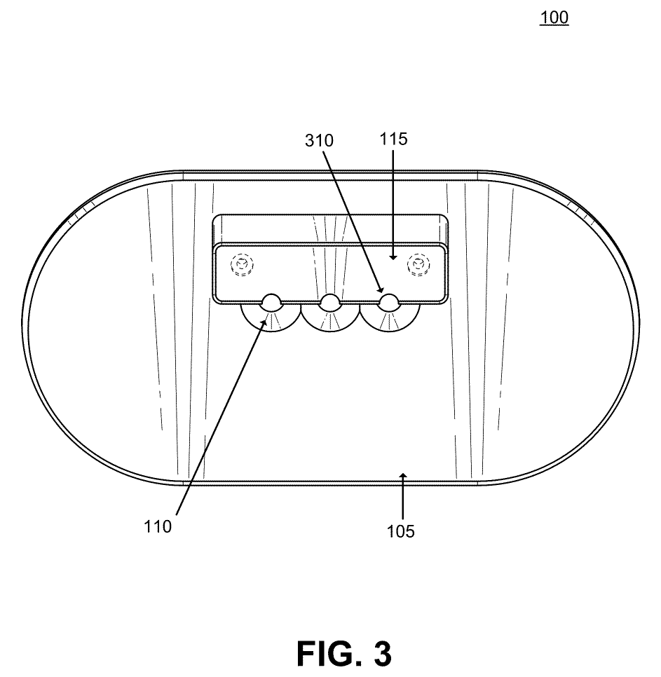

FIG. 3 illustrates a top view of the needle uncapping device 100 of FIG. 1. As shown in the displayed embodiment, the ledge 115 is substantially rectangular and includes three notches 310 corresponding to the three openings 110 positioned below the ledge 115. In embodiments in which the device 100 includes different numbers of openings, the ledge 115 includes a corresponding number of notches 310. Moreover, while the notches 310 and openings 110 in the displayed embodiment are of a uniform size, in other embodiments, the notches 310 and openings 110 are of different sizes to accommodate syringes of different types.

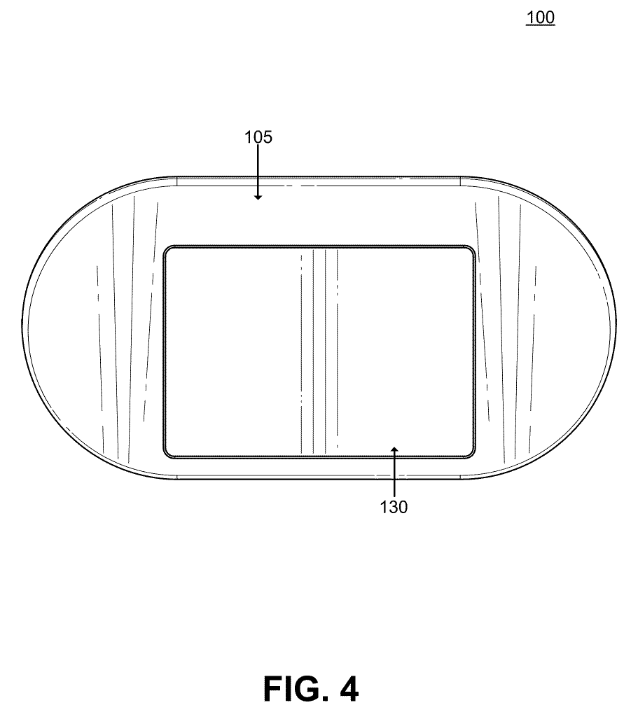

FIG. 4 illustrates a bottom view of the needle uncapping device 100 of FIG. 1. The displayed embodiment illustrates a bottom planar surface of the base 130 of the body 120. In some configurations, one or more attachment mechanisms are positioned on the bottom surface to secure the device 100 to an adjacent surface while the device is in use. The attachment mechanisms may be removable to allow the device 100 to be positioned on different surfaces. In one example, the attachment mechanism comprises one or more suction cups, thus providing increased stability while the device 100 is in use but allowing a user to detach the suction cups and move the device 100 to a different surface.

As used herein, the terms “comprises,” “comprising,” “includes,” “including,” “has,” “having,” or any other variations thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Additionally, as used herein, the term “approximately” means within 10% of the stated value unless it is otherwise apparent from the context. Furthermore, where directional terms such as above or below are used to describe the position of an element relative to another, thus should be understood to be describing the relative position of the elements when the device is under normal use. It should be appreciated that the device may be oriented differently and that other embodiments may use different orientations and that infringement of any claims does not depend on absolute orientation.

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs as disclosed from the principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A needle uncapping device comprising:
a top planar surface positioned above an elongated body configured to be held by a user of the device, the top planar surface comprising:
a rounded opening;
a ledge positioned above the top planar surface and including a lip comprising a notch positioned above the opening, the lip configured to securely engage with a lid of a needle placed in the opening;
wherein the top planar surface protrudes outward and downward from the elongated body to shield a hand of a user holding the device.

2. The needle uncapping device of claim 1, wherein the top planar surface includes a second rounded opening having a uniform size with the rounded opening, and the lip further comprises a second notch configured to securely engage with a lid of a needle placed in the second opening.

3. The needle uncapping device of claim 1, wherein the top planar surface includes a second rounded opening having a different size than the rounded opening and the lip further comprises a second notch configured to securely engage with a lid of a needle placed in the second opening.

4. The needle uncapping device of claim 3, wherein the rounded opening and the second rounded opening are color coded or otherwise visually distinguished.

5. The needle uncapping device of claim 3, wherein the notch and the second notch are of different sizes, a size of the notch being based on a size of the rounded opening and a size of the second notch being based on a size of the second rounded opening.

6. The needle uncapping device of claim 1, wherein the elongated body includes a grip on a front or back of the elongated body, the grip sized to engage with the hand of the user holding the device.

7. The needle uncapping device of claim 1, wherein the needle uncapping device is a unibody construction.

8. The needle uncapping device of claim 1, wherein the needle uncapping device is comprised of nylon, acrylonitrile butadiene styrene plastic, thermoplastic polymer, or other rigid material.

9. The needle uncapping device of claim 1, wherein the top planar surface is angled downward at an approximately 10- to 30-degree angle.

10. The needle uncapping device of claim 1, the device includes a base that protrudes outward from a front and back of the elongated body at an approximately 40- to 70-degree angle.

11. The needle uncapping device of claim 10 further comprising an attachment mechanism on a bottom surface of the base, the attachment mechanism configured to secure the device to an adjacent surface.

12. The needle uncapping device of claim 1, wherein the rounded opening has a first diameter at the top planar surface of the device and a second diameter at a base of the opening, the first diameter larger than the second diameter.
